(19) United States
(12) Patent Application Publication
(10) Pub. No.: US 2007/0178146 A1
(43) Pub. Date: Aug. 2, 2007

(54) Slow release agent absorbent dressing

(76) Inventors: Chang-Tsung Chou, Taipei City (TW); Chia-Ju Shih, Taipei City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/342,815

(22) Filed: Jan. 31, 2006

(51) Int. Cl. A61L 15/00

(52) U.S. Cl. 424/445; 442/123

(57) ABSTRACT

A slow release agent absorbent dressing particularly comprises a wound-contacting layer covered with tapered apertures. The bottom surface of said tapered apertures contacts a wound area to discharge exudate from the wound area and transmit exudate via a guiding layer to an absorbent layer. Said absorbent layer formed of high-molecular polymeric fibers is mixed with a certain concentration of water-soluble agents, such as antiseptic agents, enzymes and growth factor agents, in a suitable amount. After exudate is into the absorbent layer, said polymeric fibers expand forming into the shape of gel to avoid exudate flowing backward to the wound area. Also, a translucent evaporating layer having numerous micro pores for air venting is above the absorbent layer and the peripheral edges are joined together in the form of a sealed structure by heat-sealing to stop side escape of exudate, which is more effective in preventing the wound area from secondary infection.

### BACKGROUND OF THE INVENTION

1. Field of the Invention

The present invention relates to a slow release agent absorbent dressing and more particularly to a wound-contacting layer with tapered apertures wherein exudate discharged from a wound area is able to penetrate through to prevent from the backflow of exudate and avoid infecting the wound area. Also, mix a certain concentration of water-soluble antiseptic agents, enzymes and growth factor agents in a suitable amount into a dry absorbent layer of the dressing of the present invention, which is more effective in infection control, debridement and promoting wound-healing.

2. Prior Art

Almost every healing process generally used in the medical institution is necessarily using antimicrobial dressings to cover or bandage a wound for preventing from infection by external bacteria and the wound is possible to be the laceration, surgical incision, inflammation or burns. In addition, most commonly used dressings in the medical institution are still gauze or cotton wad. However, these dressings are effective in absorbing exudate while directly applied on the wound but exudate might flow backward to infect the wound or said absorbed exudate might evaporate to make gauze become harder; in that case, a healing process could be hindered by causing a secondary injury.

Besides, U.S. Pat. No. 6,600,085 discloses an absorbent article made from an inner core absorbent material surrounded by a top layer and a bottom layer that are apertured films. Obviously, said absorbent article is comprised of only three layers. There is a top layer, a bottom layer and an absorbent core. The structure in the prior art is different from the structure of the present invention. Also, although it discloses a bottom layer using protuberances to prevent backflow of exudate but does not contemplate a layer to disperse exudate thereby increasing the absorbing capabilities, a guiding layer provided in the present invention can overcome this disadvantage.

U.S. Pat. No. 6,333,093 discloses a wound dressing in which an anti-microbial coating can be applied on one or more of the layers of the dressing in order to provide an anti-microbial effect to limit infection. But in the present invention, the concept of providing water-soluble agents to release flowing backward to the wound area and a guiding layer as a fourth layer (in-between other layers) to enhance and optimize the passing of exudate from the wound area to the absorbent layer is a new skill to the prior art.

### OBJECTS OF THE INVENTION

The main object of the present invention is to provide a slow release agent absorbent dressing; more particularly to a wound-contacting layer formed of a one-way porous film with tapered apertures for discharging exudate from a wound area and transmitting exudate via a guiding layer to an absorbent layer to prevent from backflow and also a certain concentration of water-soluble agents mixed into a dry absorbent layer. Said certain concentration is considered by the saturation capacity of said absorbent layer. After the absorbent layer absorbs discharged exudate being in a saturated state, the certain concentration of dissolved agents will release through said porous film of the wound-contacting layer and flow backward to the wound area, which achieves the effect of producing bacteria static, lysis of necrotic tissue and even promoting granulation growth.

### BRIEF DESCRIPTION OF THE DRAWINGS

These and other features, aspects and advantages of the present invention will become apparent from the following description, claims and the accompanying embodiment shown in the drawing, which is briefly described below.

FIG. 1 is an enlarged cross-sectional view of the present embodiment of the slow release agent absorbent dressing.

FIG. 2 is a perspective view of the present embodiment of the slow release agent absorbent dressing.

### DESCRIPTION OF THE REFERRED EMBODIMENT

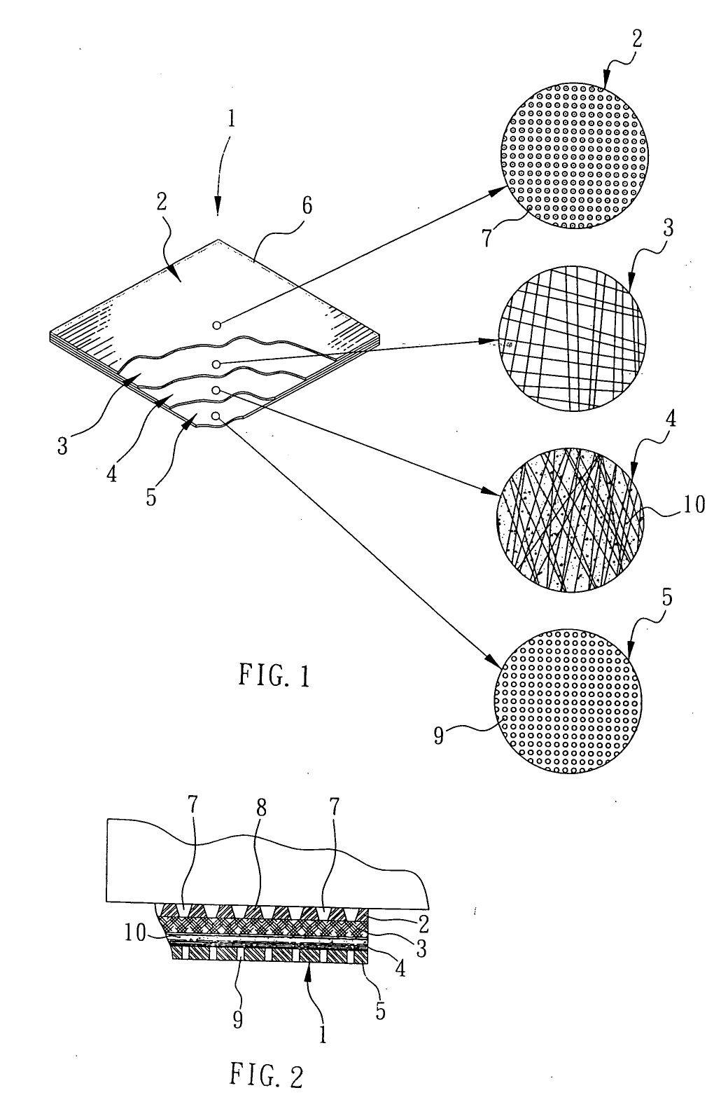

Referring to FIG. 1 and FIG. 2, the slow release agent absorbent dressing (1) of the present invention comprises a wound-contacting layer (2), a guiding layer (4), an absorbent layer (5) and an evaporating layer (6). The placement of the above layer is one on top of another in order and the peripheral edges are joined together by heat-sealing (7). The slow release agent absorbent dressing (1) has various sizes in accordance with the different size of a wound.

Wherein the wound-contacting layer (2) has a porous soft-film covered with tapered apertures (3). Said film can be a non-adherent polyethylene film or other similar materials and said tapered apertures (3) can be polygonal tapered or conical shaped apertures. The bottom surface of tapered apertures (3) with the largest caliber contacts a wound area (9) (as shown in FIG. 2) to produce a one-way valve and discharged exudate from the wound area (9) is able to penetrate through numerous tapered apertures (3). And said discharged exudate is transmitted via the guiding layer (4) to the absorbent layer (5) forming into the shape of gel to prevent from backflow. Said absorbent layer (5) is formed of high-molecule polymeric materials, such as polyester or algin mixed with the certain concentration of water-soluble agents (8). Said agents (8), can be antiseptic agents, enzymes and growth factor agents, in a suitable amount are added in a dry absorbent layer (5) and said certain concentration is considered by the saturation capacity of said absorbent layer (5). After the absorbent layer (5) absorbs discharged exudate being in a saturated state, the certain concentration of dissolved agents (8) will flow backward to the wound area (9), which achieves the effect of infective control. Therefore, as the absorbent layer (5) is mixed with antiseptic agents, the dressing (1) has the function of producing bacteria static; as the absorbent layer (5) is mixed with enzymes, the dressing (1) has the function of lysis of necrotic tissue and debridment and as the absorbent layer (5) is mixed with growth factor agents, the dressing (1) has the function of promoting granulation growth. In the present embodiment, the wound-contacting layer (2) herein is formed of a soft-film covered with tapered apertures (3).

Besides having the advantage of producing a one-way valve for discharging exudate, it is not adhering to the wound; for this reason, the dressing (1) is removed easily without hurting newborn epidermis.

As shown in FIG. 1 and FIG. 2, the guiding layer (4) joined to the upper of the wound-contacting layer (2), can be directional fine-hole fibers or non-woven fabrics to transmit said discharged exudate to the absorbent layer (5). Said absorbent layer (5) attached to the upper of said guiding layer (4) is form of high-molecule polymeric materials, such as polyester or algin. After absorbing discharged exudation, high-molecule polymeric fibers expand forming into the shape of gel to avoid exudate flowing backward to the wound area (9). Said absorbent layer (5) is added with the certain concentration of said agents (8) in a suitable amount to absorb discharged exudate. Said agents (8) can be antiseptic agents, enzymes and growth factor agents. After being in a saturated state, the certain concentration of dissolved agents (8) will flow backward to the wound area (9). In the present embodiment, non-woven pulp and high-molecule polymeric fibers under an antiseptic treatment are used. The evaporating layer (6), above the absorbent layer (4), having a translucent film with a broad spread of micro pores (10) to keep the wound area (9) in suitable moisture and people can observe the absorbing condition of the dressing (1) through said evaporating layer (6).

What is claimed is:

1. A slow release agent absorbent dressing, comprising:
a wound-contacting layer, said wound-contacting being formed of a soft-film and covered with tapered apertures;
a guiding layer, said guiding layer attached to an upper of said wound-contacting layer and being formed of directional fine-hole fibers or non-woven fabrics;
an absorbent layer, said absorbent layer attached to an upper of said guiding layer, said absorbent layer being formed of high-molecule polymeric materials, said absorbent layer absorbing exudate discharged from a wound area,
an evaporating layer, said evaporating layer attached to an upper of said absorbent layer, said evaporating layer having a translucent film and micro pores for venting air thereby keeping said wound in suitable moisture,
wherein said a guiding layer disperses said exudate from said wound-contacting layer to said absorbent layer in a substantially uniform manner to increase absorbent capabilities of the absorbent layer thereby preventing said exudate captured on said absorbent layer from passing back to said wound area.

2. A slow release agent absorbent dressing as recited in claim 1, further comprising a certain concentration of water-soluble agents mixed into said absorbent layer can be antiseptic agents, enzymes and growth factor agents and said certain concentration is based upon the saturation capacity of said absorbent layer.
